(19) United States
(12) Patent Application Publication
(10) Pub. No.: US 2004/0171689 A1
(43) Pub. Date: Sep. 2, 2004

(54) Screening method using solid supports modified with self-assembled monolayers

(76) Inventors: Pierre Desreumaux, Lille (FR); Sebastien Dharancy, Saint-Andre (FR); Johan Auwerx, Hindisheim (FR)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/474,218

(22) PCT Filed: Apr. 4, 2002
(86) PCT No.: PCT/FR02/01185

(30) Foreign Application Priority Data
Apr. 4, 2001 (FR) 01/04601

(51) Int. Cl. A61K 31/203; A61K 38/00

(52) U.S. Cl. 514/559; 514/12

(57) ABSTRACT

The invention concerns a screening method of a compound modulating the activity of a nuclear RXR-receptor heterodimer, preferably RXR-PPAR. The invention also concerns the compound capable of being selectively hybridised with the gene or a product of the gene coding for the RXR and PPAR subunits of said heterodimer, for preparing a medicine for preventive and/or curative treatment of an infection by the hepatitis C virus, or of fatty liver, of liver inflammation, liver lesions, liver cirrhosis, post-hepatic cancer whether or not associated with a hepatitis C virus infection.

The present invention relates to the field of medical biology, and more particularly to preventive and curative treatments for viral infection with the hepatitis C virus. The invention relates more particularly to a method for screening a compound modulating the activity of an RXR nuclear receptor, preferably RXR-PPAR, heterodimer. The invention also relates to the compound which can be obtained using the screening method of the invention, in particular the compounds which are agonists of the RXR-PPAR heterodimer, and also a compound capable of hybridizing selectively with the gene or a product of the gene encoding the RXR and PPAR subunits of said heterodimer, for preparing a medicinal product intended for the preventive and/or curative treatment of a hepatitis C virus infection, of fatty liver whether or not associated with a hepatocyte infection with the hepatitis C virus, of liver inflammation and of liver damage whether or not associated with a hepatocyte infection with the hepatitis C virus, and also for liver cirrhosis and/or posthepatic cancer whether or not associated with a hepatocyte infection with the hepatitis C virus.

The hepatitis C virus (HCV) has been determined as the main agent responsible for post-transfusion hepatitis, and associated with drug addiction (Kuo G, Science 1989). HCV infection represents a major public health problem since its frequency in France is estimated at 1.3% (Roudot-Thoraval F, Hepatology 1998). More than 600 000 individuals are therefore affected by this infection in our country. HCV is characterized by strong affinity for liver cells (hepatocytes), leading to acute inflammation of this organ or acute hepatitis. This acute hepatitis is most commonly asymptomatic from a clinical point of view and, in 80% of cases, progresses to chronic hepatitis which can become complicated by cirrhosis and liver cancer (Poynard T, Lancet 1997). It is estimated that 3 million patients worldwide have chronic viral hepatitis C, 20% of which will progress toward cirrhosis over a period of 10 years; the patients suffering from cirrhosis will then have an annual risk of liver cancer of 3%. Another complication of the disease is thought to be the appearance of diabetes, since recent epidemiological studies show that patients carrying HCV have an increased risk of type II diabetes (relative risk 2 to 6) (Mehta SH, Ann Intern Med 2000; Sangiorgio L, Diabetes Res Clin Pract 2000).

The treatment for chronic hepatitis C is currently based on a combination of two molecules: alpha-interferon and ribavirin (McHutchison J, N Eng J Med 1998). These medicinal products have many counter-indications and, overall, are poorly tolerated by patients. In addition, this combination of medicinal products is ineffective in approximately one patient in two despite prolonged use (6 to 12 months). A new therapeutic protocol with delayed Interferon (Schering Plough laboratory) having obtained a marketing authorization in March 2001 is going to begin in our country. The expected results allow a 10% increase in the response to treatment for a cost of 100 KF/year/patient. Faced with the lack of available vaccine and with the difficulty in effectively treating patients suffering from hepatitis C, there exists an urgent and real need to develop novel preventive and therapeutic strategies for hepatitis C.

This is precisely the subject of the present invention. The inventors have thus identified a novel therapeutic target, consisting of Peroxysome Proliferator-Activated Receptors (PPARs), for treating hepatitis C virus infections.

The identification of this novel target is based on the recent data from the literature and on results obtained by the inventors. The hepatitis C virus penetrates into hepatocytes using, in part, the low density lipoprotein receptor (LDLR or CD36) (Monazahian M, J Med Virol 1999), and then multiplies in the cell and can then invade others. This infection induces an immune response which is most commonly incapable of spontaneously eliminating the virus; this results in chronic hepatitis in approximately 80% of cases (Alter M, N Eng J Med 1992). The quality of the immune response required to eliminate the virus remains poorly understood, but the inventors have shown that the production of interleukin 4 (IL-4) constitutes a factor for poor prognosis with respect to the progression of the infection (Dharancy et al.). Other cytokines are also involved in the inflammatory mechanisms associated with hepatitis C virus infection, in particular via the production of TNFα, IL-1β and IL-18 (Nelson DR, Dig Dis Sci 1997; McGuinness PH, Gut 2000) leading to activation of the NFκB complex and to the production of molecules involved in the inflammatory reaction. These studies on the production of cytokines suggest that nuclear receptors might be involved in regulating the immune response during hepatitis C virus infections. Thus, it has been demonstrated that IL-4 is the main cytokine regulating the expression of PPARγ, which plays an essential role in regulating the production of inflammatory cytokines such as TNFα and IL-1β via inhibition of NFκB.

Hepatitis C infection is also responsible for a characteristic fatty overload of the hepatocytes, referred to as fatty liver (Goodman Z D, Semin Liver Dis 1995; Czaja A, J Hepatol 1998; Clouston AD, J Hepatol 2001) which constitutes the first clinical manifestation of hepatitis C virus infection. The cause of this fatty liver is unknown, but may be secondary to a modulation of the activation of nuclear receptors, which represent one of the largest families of transcription factors, some of which can be activated by small lipophilic molecules such as hormones and nutrients for example. An important subfamily of these nuclear receptors corresponds to the factors which have the property of forming a heterodimer with the retinoid X receptor (RXR). This subfamily is composed in particular of the vitamin D receptor (VDR), of the retinoic acid receptor (RAR), of the thyroid hormone receptor (TR), of the peroxysome proliferator-activated receptors (PPARs), of the bile acid receptors (BARs, also referred to as FXRs), and of the oxysterol receptors (LXRs), and steroid and xenobiotic receptors (SXRs, also referred to as PXRs).

The peroxysome-activated receptors (PPARs) are nuclear receptors capable of forming a heterodimer with RXR. 4 types of PPAR exist: α, β, γ and δ. PPARα expression is mainly located in the liver and muscle and in a certain form of adipose tissue corresponding to brown fat. The expression of PPARδ or PPARβ is ubiquitous, whereas PPARγ is produced essentially in the adipose tissue, the macrophages and the colonic epithelial cells. PPARγ production in the liver in humans remains unknown. These PPAR receptors are involved in the transcription of many target genes after binding to a specific response element (PPRE) of the DNA. Various synthetic and natural ligands can bind PPAR and activate it. The activation of PPAR brings about various biological effects, including the activation, differentiation and proliferation of fat cells (adipocytes), regulation of lipid metabolism, regulation of inflammation and regulation of the response to insulin (Lehmann J M, J Biol Chem 1995) (Shoonjans K, Biochim Biophys Acta 1996). Other receptors which heterodimerize with RXR, such as the LXRs, TRs, BARs/FXRs and SXRs/PXRs, are highly expressed by hepatocytes. The LXR and FXR/BAR receptors probably play an important role in liver homeostasis since they control the metabolism of cholesterol and of bile acids, and there exists a regulation between these receptors and PPAR. Thus, PPARs are capable of inducing the expression of LXRα in the liver (Tobin & Auwerx, Mol. Endo 2000) and macrophages (Tontonoz & Evans, Mol. Cell, 2001).

The inventors therefore propose using PPARα, -β, -δ and -γ, PPAR modulators, and also the nuclear receptors capable of forming a heterodimer with RXR (LXR, TRs, BAR/FXR and SXR/PXR) as a novel therapeutic target and/or novel therapeutic proteins for treating viral hepatitis C, by acting on the penetration of the virus into the cells, by decreasing the inflammatory reaction and the fibrosis and/or by decreasing the risk of cancer.

More particularly, the present invention relates to a method for screening a compound modulating the activity of an RXR-nuclear receptor heterodimer, characterized in that said method comprises the steps of (1) bringing said RXR-nuclear receptor heterodimer into contact, in the presence of the reagents required to carry out at least one transcription reaction, with at least one reporter gene having all the genetic information required for the expression of a reported protein, said gene having at least one RE regulatory sequence; (2) bringing said RXR-nuclear receptor heterodimer and said compound into contact, in the presence of the reagents required to carry out at least one transcription reaction, with at least one reporter gene having all the genetic information required for the expression of a reporter protein and which has at least one RE regulatory sequence; (3) qualitatively, optionally quantitatively, determining the expression of said protein treated in steps 1) and 2) and then comparing said expressions; finally, (4) selecting, optionally identifying, the compound if said compound is capable of selectively modulating the expression of said protein.

The expression “activity of the heterodimer” is intended to denote the natural biological activity or the natural biological function performed by the heterodimer in a live cell, i.e. a transcription factor activity. For the purpose of the present invention, a transcription factor is a diffusible protein factor capable of positively or negatively modulating the expression of one or more genes by interacting with their regulatory sequence(s). Preferably, said RXR-nuclear receptor heterodimer is selected from the group composed of RXR-PPAR, RXR-LXR, RXR-TR, RXR-BAR/FXR and RXR-SXR/PXR. Among the SXR steroid receptors, mention should in particular be made of glucocorticoid receptors (GRs), estrogen receptors (ERs), mineralo-corticoid receptors (MRs), and the progesterone receptor (PR). The term “nuclear receptors” is intended to denote receptors which have an identical overall structure, namely: (1) an NH₂-terminal end which is variable in length, which is not conserved and which is specific to the receptor (A-B domain of the gene); (2) a very conserved region of approximately 65 amino acids (C domain of the gene) which interacts with the DNA; this region contains two zinc-finger motifs; (3) a nonconserved region of variable length (D domain); and (4) a domain of variable length which corresponds to the region where the hormone or the ligand binds (E domain).

For the purpose of the present invention, the expression “RE (for “responsive element”) regulatory sequence” is intended to denote nucleic acid sequences required for gene regulation in eukaryotes. These sequences differ from the promoter sequence in the strict sense, which corresponds to the sequence where RNA polymerase II binds. The RE regulatory sequences are generally located in the 5′ upstream region of genes, upstream of the promoter sequence. They constitute CIS-regulatory sequences to which TRANS-acting transcription factors bind. In general, these RE sequences confer tissue specificity on the genes which possess them. Preferably, said RE regulatory sequence is selected from the group composed of the PPAR-responsive regulatory sequence PPRE, the LXR-responsive regulatory sequence LX-RE, the thyroid hormone receptor (TR)-responsive regulatory sequence TRE, the BAR/FXR-responsive regulatory sequence BAR/FXR-RE, and the SXR/PXR-responsive regulatory sequence SXR/PXR-RE. Among the steroid receptor-REs (SXR-REs), mention should in particular be made of the glucocorticoid receptor RE (GRE) regulatory sequence, the estrogen receptor RE (ERE) regulatory sequence, the mineralocorticoid receptor RE (MRE) regulatory sequence, and the progesterone receptor RE (PRE) regulatory sequence.

More preferably, the heterodimer according to the invention is RXR-PPAR and the RE (responsive element) regulatory sequence is PPRE.

All the genetic information required for a transcription reaction is known to those skilled in the art; it involves at least one promoter, transcription initiation and termination signals, and also suitable transcription-regulating regions. Optionally, the reporter gene transcribed can be translated. For this, it is essential for the mRNA to possess translation initiation and termination signals.

The reporter gene may be present in linear form, it is preferably cloned into an expression vector allowing expression of the reporter protein in a cellular host. The vector may or may not be maintained stably in the cell and can optionally possess particular signals specifying secretion of the translated polypeptide. The various controlling signals are chosen as a function of the cellular host used. Such vectors will be prepared according to the methods commonly used by those skilled in the art, and the clones resulting therefrom can be introduced into an appropriate host by standard methods such as, for example, transfection by calcium phosphate precipitation, lipofection, electroporation or thermal shock.

The compound obtained using the screening method activates or increases the expression of said reporter gene. Alternatively, the compound obtained using the screening method preferably abolishes or inhibits the expression of said reporter gene.

According to a preferred embodiment of the invention, the screening method is characterized in that at least one step is carried out in a live cell. According to a preferred embodiment, at least steps 1 and 2 of the method are carried out in a live cell. Among the cells in which the screening method according to the invention is carried out, mention should be made of prokaryotic cells, such as bacteria and in particular Escherichia coli, yeasts, in particular Saccharomyces cerivisiae, and animal cells. Preferably, they are mammalian cells preferably chosen from human, mouse, rat, rabbit and hamster cells.

It also falls within the scope of the invention to carry out the screening of compounds of the invention on laboratory animals or using cells derived from laboratory animals. These laboratory animals, preferably mice, rats or rabbits, are preferably genetically modified so as to express a transgene which has all the genetic information required for the expression of a reporter protein, said gene having at least one RE regulatory sequence, preferably PPRE. This transgene may be present in the genome of at least one cell of the transgenic animal, this integration being carried out either by homologous recombination or by random integration. Alternatively, the transgene is present in an episomal form.

Alternatively, the method may be an in vitro screening method, characterized in that the steps are carried out in an acellular system. In this case, this method is carried out in the presence of reagents required to carry out at least one transcription reaction and, optionally, in the presence of reagents required to carry out at least one reaction of translation of the reporter protein, such as, for example, rabbit reticulocyte lysate. These various in vitro methods are well known to those skilled in the art (Sambrook et al., 1989).

In the screening method according to the present invention, any conventional procedure or assay can be used, in step 3 of said method, to obtain a detectable and/or quantifiable signal representative of the amount of said expression product present in the reaction medium, according to the expression product being sought. Preferably, the expression product is a polypeptide corresponding to the product of translation of the mRNA corresponding to said reporter gene; in this case, this expression product is revealed and, optionally, quantified by the “Western blotting” method or by immunohistochemistry, well known to those skilled in the art. When it is a transcription product (mRNA) encoding said reporter gene, it can be revealed, optionally quantified, by RT-PCR, by Northern blotting or by the “in situ hybridization” method, also well known to those skilled in the art.

According to a preferred embodiment, said reporter protein is selected from the group of autofluorescent proteins and enzymes detectable by a histochemical process. Preferably, the autofluorescent protein is chosen from the group composed of green fluorescent protein (GFP), enhanced green fluorescent protein (EGFP), red fluorescent protein (RFP), blue fluorescent protein (BFP) and yellow fluorescent protein (YFP), and fluorescent variants of these proteins. Alternatively, said reporter gene encodes an enzyme detectable by a histochemical process, chosen from the group composed of β-galactosidase, β-glucoronidase, alkaline phosphatase, glucose oxidase, glucose amylase, carbonic anhydrase, acetylcholine esterase, lysozyme, malate dehydrogenase, glucose-6-phosphate dehydrogenase, alcohol dehydrogenase, luciferase, chloramphenicol acetyltransferase, and growth hormone. More generally, said reporter gene may correspond to any gene which has at least one PPRE upstream regulatory sequence. This may be a gene present in its natural environment or a gene genetically manipulated in order to place a coding sequence under the control of a functional promoter sequence comprising at least the RE sequence, preferably PPRE.

The present invention also relates to the compound which can be obtained using the screening method according to the invention and also to any compounds modulating the activity of an RXR-nuclear receptor heterodimer.

According to a first embodiment, this compound activates or increases the activity of the RXR-nuclear receptor heterodimer, in particular RXR-PPAR.

Preferably, the compound is selected from the group composed of PPARα, PPARβ, PPARγ, PPARδ and RXR, and a ligand which is a natural or synthetic agonist of PPARα, PPARγ, PPARβ, PPARδ or RXR, or of an RXR-nuclear receptor, preferably of RXR-PPAR, and in particular RXR-PPARα, RXR-PPARβ, RXR-PPARγ and RXR-PPARδ. This ligand which is an agonist of RXR-PPAR is preferably chosen from the group composed of the J2 prostaglandins, polyunsaturated fatty acids, thiazolinediones, nonsteroidal anti-inflammatories, fibrates, in particular fenofibrate, pioglitazone. Among the agonist compounds, mention should also be made of anti-ideotype antibodies corresponding to the PPARα, PPARβ, PPARγ, PPARδ and RXR compounds.

According to a second embodiment, the compound which can be obtained using the screening method according to the invention abolishes or inhibits the activity of the RXR-nuclear receptor heterodimer. Preferably, it is a ligand which is a natural or synthetic antagonist. The term “antagonist ligand” is intended to denote the compounds capable of decreasing or abolishing the level of expression and/or the activity of RXR-nuclear receptor, in particular RXR-PPAR. The term “antagonist” refers to a molecule which, when it binds to the polypeptide according to the invention, decreases the amount or the duration of the effects of the biological activity of the RXR-PPAR polypeptide. Among the compounds which can be obtained using the screening method according to the invention and which abolish or inhibit the activity of the RXR-nuclear receptor heterodimer, mention may be made of the polynucleotides capable of hybridizing specifically with the RE, preferably PPRE. Thus, for example, said polynucleotide competes with the RXR-PPAR heterodimer for binding to the PPRE responsive element. The polynucleotide may also correspond to the RE sequence, preferably PPRE in excess, in order to bind, by excess of substrate, the RXR-nuclear receptor heterodimer, preferably RXR-PPAR, on the RE sequence, preferably PPRE, of the polynucleotide. The polynucleotide may also correspond to an antisense oligonucleotide. In this case, the polynucleotide is capable of hybridizing selectively with the gene or a product of the gene encoding the RXR and PPAR subunits. The oligonucleotides according to the invention have a minimum size of 9 bases, preferably of at least 10, 12, 15, 17, 20, 25, 30, 40, 50 bases. According to yet another embodiment, this compound may be a monoclonal or polyclonal antibody, or antibody fragment, directed specifically against the RXR-nuclear receptor, preferably RXR-PPAR; preferably, the antibody according to the invention is directed against the PPAR subunit of the heterodimer, it is preferably a subunit of the PPARγ type. Among the antibodies according to the invention, mention may be made of the rabbit polyclonal antibodies directed against PPARγ and marketed by WAK-CHEMIE (Bad Soden, Germany) or by TEBU (Le Perray en Yvel-ines, France). In general, for the preparation of monoclonal antibodies or fragments thereof directed against the PPARγ receptor, reference may be made to the techniques which are in particular described in the manual “Antibodies” (Harlow et al., 1988) or to the preparation technique using hybridomas, described by Kohler and Milstein in 1975. The monoclonal or polyclonal antibodies according to the invention can be obtained, for example, from a cell of an animal immunized against the PPARγ protein, or a fragment thereof comprising the specific epitope (determinant of the protein responsible for the specific interaction with the antibody). Said PPARγ receptor protein, or a fragment thereof, may in particular be produced, according to the usual procedures, by genetic recombination using a nucleic acid sequence contained in the cDNA sequence encoding the PPARγ receptor protein, or by peptide synthesis. The monoclonal or polyclonal antibody fragments according to the invention comprise any fragment of said monoclonal antibody capable of binding to the epitope of the PPARγ protein to which the monoclonal or polyclonal antibody from which said fragment is derived binds. Examples of such fragments include in particular single-chain monoclonal or polyclonal antibodies or Fab or Fab′ monovalent fragments and divalent fragments such as F(ab′)2, which have the same binding specificity as the monoclonal antibody from which they are derived. A fragment according to the invention may also be a single-chain Fv fragment produced by methods known to those skilled in the art and as described, for example, by Skerra et al., 1988 and King et al., 1991. A fragment according to the invention may also be an Fc fragment. The monoclonal or polyclonal antibody fragments of the invention can be obtained from the monoclonal or polyclonal antibodies as described above by methods such as digestion with enzymes, for instance pepsin or papain, and/or by cleavage of the disulfide bridges by chemical reduction. Alternatively, the monoclonal or polyclonal antibody fragments can be synthesized by automatic peptide synthesizers such as those provided by the company Applied Biosystems, etc., or can be prepared manually using techniques known to those skilled in the art and as described, for example, by Geysen et al. (1978).

The agonist and antagonist ligands include proteins, nucleic acids, carbohydrates, lipids and chemical compounds. Among the agonist and antagonist ligands which are protein in nature, all the natural polypeptides capable of interacting with the RXR-nuclear receptor heterodimer, preferably RXR-PPAR, should be denoted.

The term “ligand” or “compound” is intended to define all the compounds capable of interacting directly or indirectly with the binding of the RXR-nuclear receptor heterodimer, preferably RXR-PPAR to the RE sequence, preferably PPRE; for the purpose of the present invention, a ligand forms a complex which affects the transcriptional activity, i.e. increases, decreases, modulates or knocks out the transcription of a gene under the control of a promoter containing an RE DNA sequence to which the RXR-nuclear receptor heterodimer binds.

One of the objects of the invention is also to provide a kit or pack for screening ligands capable of affecting the transcriptional activity of a reporter gene the promoter sequence of which comprises at least one RE sequence, preferably PPRE, capable of binding an RXR-nuclear receptor heterodimer, preferably RXR-PPAR, and characterized in that it comprises the following elements: (i) a gene, optionally cloned into an expression vector, and optionally present in a live cell; (ii) a ligand; (iii) the reagents required for carrying out a transcription and/or translation reaction.

According to another aspect, the compound or ligand according to the invention is used as a medicinal product, and in particular as active principles of a medicinal product for humans or animals. The compound is preferably in soluble form, combined with a pharmaceutically acceptable vehicle. The expression “pharmaceutically acceptable vehicle” is intended to denote any type of vehicle normally used in the preparation of injectable compositions, i.e. a diluent, a suspending agent such as an isotonic or buffered saline solution. Preferably, the compound is administered systemically, in particular intravenously, intramuscularly, intradermally or orally. Their optimal modes of administration, dosages and pharmaceutical forms can be determined according to the criteria generally taken into account in establishing a treatment suitable for a patient, such as, for example, the age or bodyweight of the patient, the seriousness of his or her general condition, the tolerance to the treatment and the side effects observed, etc.

The present invention also relates to the use of a compound modulating the activity of an RXR-nuclear receptor heterodimer, for preparing a medicinal product intended for the preventive and/or curative treatment of an infection with a virus which uses, in order to perform its viral cycle, at least one cellular protein encoded by a gene which has at least one RE regulatory sequence. Preferably, said cellular protein is the low density lipoprotein receptor (LDLR or CD36) and said virus is the hepatitis C virus.

Preferably, said RXR-nuclear receptor heterodimer is selected from the group composed of RXR-PPAR, RXR-LXR, RXR-TR, RXR-BAR/FXR and RXR-SXR/PXR. According to a preferred embodiment, said RE regulatory sequence is selected from the group composed of the PPAR-responsive regulatory sequence PPRE, the LXR-responsive regulatory sequence LX-RE, the thyroid hormone receptor (TR)-responsive regulatory sequence TRE, the BAR/FXR-responsive regulatory sequence BAR/FXR-RE, and the SXR/PXR-responsive regulatory sequence SXR/PXR-RE; preferably, it is PPRE. Preferably, said RXR-nuclear receptor heterodimer is RXR-PPAR and said RE regulatory sequence is PPRE.

The invention also relates to the use of a compound modulating the activity of an RXR-nuclear receptor heterodimer, for preparing a medicinal product intended for the preventive and/or curative treatment of an infection, with the hepatitis C virus, of cells selected from hepatocytes, bile cells, liver parenchymal cells, and circulating blood cells.

The invention also relates to the use of a compound modulating the activity of an RXR-nuclear receptor heterodimer, for preparing a medicinal product intended for the preventive and/or curative treatment of chronic hepatitis.

The invention also relates to the use of a compound modulating the activity of an RXR-nuclear receptor heterodimer, for preparing a medicinal product intended for the preventive and/or curative treatment of fatty liver whether or not associated with a hepatocyte infection with the hepatitis C virus.

The invention also relates to the use of a compound modulating the activity of an RXR-nuclear receptor heterodimer, for preparing a medicinal product intended for the preventive and/or curative treatment of liver inflammation and of liver damage whether or not associated with a hepatocyte infection with the hepatitis C virus.

The invention also relates to the use of a compound modulating the activity of an RXR-nuclear receptor heterodimer, for preparing a medicinal product intended for the preventive and/or curative treatment of liver cirrhosis and/or of a posthepatic cancer whether or not associated with a hepatocyte infection with the hepatitis C virus.

The invention also relates to the use of a compound modulating the activity of an RXR-nuclear receptor heterodimer, for preparing a medicinal product intended for the control of carbohydrate metabolism and/or for the preventive and/or curative treatment of type II diabetes in patients carrying the hepatitis C virus.

The present invention also relates to the use of a compound according to the invention or of a compound capable of hybridizing selectively with the gene or a product of the gene encoding RXR and/or PPAR, for preparing a medicinal product intended for the preventive and/or curative treatment of an infection with a virus which uses, in order to perform its viral cycle, at least one cellular protein encoded by a gene which exhibits at least one RE regulatory sequence, preferably PPRE. Preferably, said cellular protein is the low density lipoprotein receptor (LDLR or CD36) and said virus is the hepatitis C virus.

In addition, the present invention relates to the use of a compound according to the invention or of a compound capable of hybridizing selectively with the gene or a product of the gene encoding RXR and/or PPAR, for preparing a medicinal product intended for the preventive and/or curative treatment of a hepatocyte infection with the hepatitis C virus.

The present invention also relates to the use of a compound according to the invention or of a compound capable of hybridizing selectively with the gene or a product of the gene encoding RXR and/or PPAR, for preparing a medicinal product intended for the preventive and/or curative treatment of fatty-liver whether or not associated with a hepatocyte infection with the hepatitis C virus.

The present invention also relates to the use of a compound according to the invention or of a compound capable of hybridizing selectively with the gene or a product of the gene encoding RXR and/or PPAR, for preparing a medicinal product intended for the preventive and/or curative treatment of liver inflammation and of liver damage whether or not associated with a hepatocyte infection with the hepatitis C virus.

The present invention also relates to the use of a compound according to the invention or of a compound capable of hybridizing selectively with the gene or a product of the gene encoding RXR and/or PPAR, for preparing a medicinal product intended for the preventive and/or curative treatment of liver cirrhosis and/or of a posthepatic cancer whether or not associated with a hepatocyte infection with the hepatitis C virus. The invention also relates to a product comprising at least one compound according to the invention or a compound capable of hybridizing selectively with the gene or a product of the gene encoding RXR and/or PPAR and at least one anticancer agent, as a combination product for simultaneous or separate use or use spread out over time, in anticancer therapy.

The present invention also relates to the use of a compound according to the invention or of a compound capable of hybridizing selectively with the gene or a product of the gene encoding RXR and/or PPAR, for preparing a medicinal product intended for the control of carbohydrate metabolism and/or for the preventive and/or curative treatment of type II diabetes in patients carrying the hepatitis C virus.

Finally, the present invention relates to the use of a composition comprising a compound according to the invention modulating the activity of an RXR-nuclear receptor heterodimer, preferably RXR-PPAR, and a pharmaceutically acceptable vehicle, as a medicinal product for the preventive and/or curative treatment of a human being or of an animal infected with the hepatitis C virus, characterized in that the ability of said compound to selectively modulate the activity of said heterodimer is determined by (a) bringing said heterodimer into contact, in the presence of the reagents required to carry out at least one transcription reaction, with at least one gene having all the genetic information required for the expression of a protein, said gene having at least one RE regulatory sequence, preferably PPRE; (b) bringing said heterodimer and said compound into contact, in the presence of the reagents required to carry out at least one transcription reaction, with at least one gene having all the genetic information required for the expression of a protein and which has at least one RE regulatory sequence, preferably PPRE; (c) qualitatively, optionally quantitatively, determining the expression of said protein triggered in (a) and (b) and then comparing said expressions; (d) finally, identifying the compound which selectively modulates the expression of said protein.

The invention also relates to a pharmaceutical composition for the preventive and/or curative treatment of a hepatitis C virus infection, characterized in that it contains a therapeutically effective amount of a compound according to the invention or of a compound capable of hybridizing selectively with the gene or a product of the gene encoding RXR and/or PPAR. This composition may also contain at least one antiviral agent as a combination product for simultaneous or separate use or use spread out over time, in antiviral therapy associated with a hepatitis C virus infection; this antiviral agent is preferably selected from the group composed of alpha-interferon (αIFN), ribavirin and delayed interferon.

Other characteristics and advantages of the invention will emerge in the remainder of the description with the examples represented hereinafter. In these examples, reference will be made to the following figures.

### FIGURES

FIG. 1: Demonstration of the expression of PPAR nuclear receptors in hepatic cells from healthy individuals (controls) and from patients infected with the hepatitis C virus (HCV). Quantification by competitive RT-PCR of PPARγ expressed in number of PPARγ mRNA molecules per β-actin mRNA molecules in hepatic cells of 10 controls and of 21 patients suffering from chronic hepatitis C. The mean is represented and the standard deviations are indicated.

FIG. 2a: Hepatic expression of RXRα, VDR, LXRα, PPARα, PPARδ and PPARγ messenger RNAs in samples of human livers taken from patients having chronic hepatitis C and from control individuals, using the semi-quantitative RT-PCR technique. The results are expressed in means±standard deviation relative to the healthy control livers.

FIG. 2b: Hepatic concentration of the PPARα mRNA in samples of human livers taken from patients suffering from chronic hepatitis C and from healthy controls, using the competitive RT-PCR technique. The results are expressed in means±standard deviation relative to the healthy control livers.

FIG. 3: Hepatic expression of PPARα in samples of human livers taken from patients exhibiting HCV-induced cirrhosis (n=3), HBV-induced cirrhosis (n=2), alcoholic cirrhosis (n=5), and from healthy controls (n=5), using the Western blotting technique. The results are expressed in means±standard deviation.

FIG. 4: Expression of PPARα in human peripheral blood mononuclear cells (PBMCs) taken from the patients suffering from chronic hepatitis C, and from healthy controls, using the competitive RT-PCR technique (4a) and the Western blotting technique (4b). The results are expressed as means±standard deviation relative to the healthy control livers.

FIG. 5: Susceptibility of PPARγ^{+/-} mice to CCl₄-induced acute hepatitis. The necrotic-inflammatory scores (A) are expressed as mean±standard deviation. The intensity of the lesions is evaluated by histological analysis of the livers of mice killed two days after induction of hepatitis by administration of CCl₄ (1 μm/kg) using the Ishak score. The mortality (B) is expressed as percentage lethality two days after the administration of CCl₄.

FIG. 6: Susceptibility of PPARα^{-/-} mice to CCl₄-induced acute hepatitis. The necrotic-inflammatory scores (A) are expressed as mean±standard deviation. The intensity of the damage was evaluated by histological analysis using the Ishak score on livers of mice killed 5 days after induction of hepatitis by administration of CCl₄ (1 μm/kg). The mortality (B) is expressed as percentage lethality 5 days after administration of CCl₄.

FIG. 7: Quantification of the PPARα messenger RNA in clones N3, N4 and SWX, using competitive RT-PCR. The results show a level of inhibition of PPARα expression of 90% for clone N4 (a) and of 23% for clone N3 (b). The results are represented as the mean ± standard deviation.

### EXAMPLES

1. Materials and Methods

1.1 Quantification of PPARs and other NRs Forming a Heterodimer with RXR in the Liver by PCR:

The quantification by RT-PCR of PPARs and other nuclear receptors forming a heterodimer with RXR was carried out on liver biopsies. All the biopsies had an equivalent size and the average weight was 8±4 mg. The biopsies were immediately frozen and stored at −80° C. After incubation of the samples in Trizol, the nucleic acids were extracted using chloroform. Following treatment for 30 min at 37° C. with 20-50 units of RNase-free DNase I, the total RNAs were reverse-transcribed into cDNA as previously described. The reaction mixture derived from the RT was amplified by PCR using sense and antisense primers specific for these receptors and for β-actin. The samples were subjected to 20-40 PCR cycles and the quantification of the cDNA was carried out after electrophoresis on a 3% agarose gel. Since the fine quantification of RNA in samples is often difficult and imprecise, the number of nuclear receptor mRNA molecules was expressed by comparison with the number of molecules of mRNA of an internal control, β-actin, in the same sample.

1.2 Quantification of PPARs and other Nuclear Receptors (NRs) Forming a Heterodimer with RXR in the Liver by Western Blotting:

Total protein extracts were obtained by homogenization of liver biopsies in a lysis buffer consisting of PBS with 1% NP-40, 0.5% sodium deoxycholate, 0.1% sodium dodecyl sulfate and a conventional cocktail of protease inhibitors. The separation of the total proteins (50μ), the transfer onto PVDF membrane and the immunodetection of the PPARs and other NRs forming a heterodimer with RXR by incubation of the membrane with a rabbit polyclonal antiserum for 12 hours (1/500 dilution, TEBU, Le Perray en Yvelines, France) were carried out as previously described. The complexe was revealed by chemiluminescence (ECL, Amersham, UK). The results are expressed in optical density (OD) units per 50 ng of total proteins.

1.3 Immunolabeling of PPARs and other NRs Forming a Heterodimer with RXR in the Liver:

Liver biopsies were fixed in 4% paraformaldehyde, embedded in paraffin and sectioned at 4 micrometers for immunohistochemistry and immunofluorescence. The sections were preincubated for 30 minutes at ambient temperature in a blocking solution containing avidin D and biotin (Blocking Kit, SP2001, Vector Laboratories, Burlingame, Calif., USA). They were then exposed to a specific antibody directed against PPARs or other NRs forming a heterodimer with RXR, for 2 hours at ambient temperature. The sections were washed in PBS containing 0.05% Triton X100 and incubated with a biotinylated anti-rabbit goat secondary antibody (1/500 dilution for 30 minutes, Dako, Trappes, France). The immunocomplex was detected by virtue of avidin-biotin coupled to peroxidase (ABCOMPLEX/HRP, Dako, Trappes, France) and revealed using 3,3′-diaminobenzidine (DAB, Dako, Trappes, France). As a negative control, the primary antibody was replaced with an irrelevant rabbit serum.

1.4 Patients and Collection of Liver and Blood Specimens

The study was approved by the local Ethics Committee and all the subjects gave their informed consent. The liver biopsies were carried out on 20 patients suffering from chronic hepatitis C (7 women, 13 men; average age 43±12, range 21 to 70 years old).

Chronic hepatitis C is defined by the presence of biochemical abnormalities (mean level of AST (aminotransferase) in the serum: 94±61 U/l), a positive enzyme immunoassay (EIA) for the hepatitis C virus, the presence of HCV RNA in the serum detected by PCR (mean viral load: 603 330±480 000 copies/ml), and by consistent histological data (mean histological activity index: 6.5±2.4). As controls for inflammation, the liver biopsies were treated under the same conditions when from patients suffering from chronic hepatitis B (n=3), and when from patients suffering from alcoholic cirrhosis (n=5). The surgical biopsies of histologically normal livers were obtained by hepatectomy in 14 patients operated on for liver metastases. The patients and the controls received no treatment (antiviral therapy, hepatotoxic drugs, corticosteroids, immunosuppressive treatment) before or during the study, and none of them consume more than 20 g of alcohol per day.

All the biopsy specimens were treated under the same conditions and all the samples had a similar size and an average weight of 4±1 mg. The biopsies were cut into two parts. They were immediately frozen in liquid nitrogen and stored at −80° C. for nuclear receptor messenger RNA and protein analyses. The peripheral blood mononuclear cells (PBMCs) were isolated from heparinized fresh blood obtained from 10 informed healthy volunteers and 10 patients suffering from chronic hepatitis C. After elimination of the platelet-rich plasma, the blood was centrifuged on Ficoll-Hypaque for 20 minutes at 4° C., and the mononuclear cells at the interface were collected, washed twice, then resuspended, and stored at −80° C. for the nuclear receptor mRNA and protein analyses.

1.5 Cells Expressing the Hepatitis C Virus (HCV) Core Protein

The transfected human hepatocellular carcinoma cell line (HepG2) is used to analyze the impact of the HCV core protein on the expression of PPARα. The HepG2 cells were transfected as described previously (Barba et al. Proc. Natl. Acad. Sci. USA 1997, 94: 1200-1205) with the vector pEF352 neo comprising, under the control of the elongation factor 1α promoter, an HCV cDNA including the HCV 1b sequences of the core up to the NS3 region. Two independent clones stably expressing the HCV core protein were analyzed (clone N3 and N4). The clone transfected with the empty vector was used as a negative control (clone SWX). These various clones were cultured at 37° C. under an atmosphere comprising 5% CO₂, and maintained in a Dulbecco's modified Eagle's culture medium (DMEM) supplemented with 10% fetal calf serum and comprising penicillin-streptomycin. The cells were cultured until confluency and then the culture plates were scraped and the cells were harvested so as to prepare the cell extracts.

1.6 Induction of CCl₄-Induced Hepatitis in the Mice

The experiments with animals were carried out in an approved establishment (No. B59-108 and B67-218-5) according to government directives No. 86/609/EEC. The animals were grouped into cages (5 to 6 animals per cage) and had free access to water and to rodent food. For a study of a CCl₄-induced acute pathology, the mice were anesthetized for 10 minutes and were given an intraperitoneal injection of a 1:1 solution of CCl₄ and sterile mineral oil at a dose of 1 ml/kg of animal. The control mice are given a dose of mineral oil using the same technique. First, PPARα−/− and PPARγ+/− mice, both on a 129/Sv genetic background, are used (the homozygous PPARγ−/− mouse is not viable) to verify the potential susceptibility to the development of hepatitis in these knock-out animals. In a second type of experiment, wild-type male Balb/c mice were used in studies of intervention with the PPARγ agonist pioglitazone (50 mg/kg). This compound was administered once a day by oral gavage, beginning 3 days before the induction of hepatitis. The animals were killed by cervical dislocation between days 2 and 5 after adminstration of CCl₄. The livers were recovered and then fixed overnight in 4% paraformaldehyde and then embedded in paraffin. The sections were stained with hematoxylin and eosin and then examined blind by a pathologist and then evaluated according to the Ishak score (Ishak, et al. J. Hepatol. 1995, 22: 696-699).

1.7 Quantification of the Nuclear Receptor and β-Actin Messenger RNAs by RT-PCR

The RNA was isolated from a sample of liver and mononuclear cells with the TRIzol® reagent (Life Technologies, Cergy Pontoise, France) as described by the manufacturer. After treatment at 37° C. for 30 minutes with 20 to 50 units of RNAse-free DNase I (Roche Diagnostics Corporation, Indianapolis, USA), the total RNA (10 μg) was reverse transcribed into cDNA. The reverse transcription reaction mixture was amplified by PCR using sense and antisense primers specific for the nuclear receptors (RXRα, LXRα, VDR, PPARα, PPARβ, PPARγ) and for β-actin. The samples were subjected to 40 PCR cycles (Perkin- Elmer Corporation, Foster City, Calif., USA). The cDNA is quantified by electrophoresis on a 2 to 3% agarose gel using an image analyzer (Gel Analyst, Clara Vision, Paris, France). A precise quantification of the RNA in the sample is often difficult and imprecise; the number of PPARα messenger RNA molecules in the livers is expressed by comparison with the number of 10⁶ molecules of mRNA of an internal control, in this case β-actin, in the same sample.

1.8 Quantification of PPARα by Western Blotting Analysis

The proteins were prepared from liver biopsies and from mononuclear cells as previously described. The total protein extracts were obtained by homogenization of tissues and of cells in an extraction buffer composed of PBS (phosphate buffered saline) with 1% NP-40, 0.5% sodium deoxycholate, 0.1% sodium dodecyl sulfate and a conventional cocktail of protease inhibitors. The total proteins were then separated by polyacrylamide gel electrophoresis and then electrotransferred. The immunodetection with a second antibody conjugated to peroxidase and with chemiluminescence is performed according to the manufacturer's instructions (ECL, Amersham, UK). The results are expressed as optical density units per 50 μg of total proteins.

1.9 Statistical Methods

The comparison of the mean±standard deviation for the PPARγ messenger RNAs and proteins between patients with ulcerative colitis (UC) and Crohn's disease (CD) and the controls was analyzed by the Kruskal-Wallis nonparametric one-way ANOVA test. The differences are statistically significant if p is <0.05.

2. Demonstration of the Nuclear Receptor Expression in the Liver

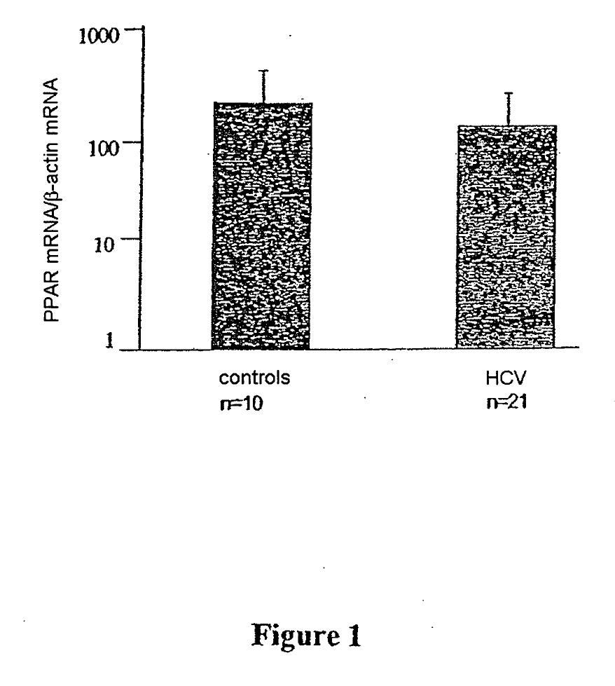

Using PCR techniques, the inventors demonstrated, in 10 controls and 21 patients suffering from chronic viral hepatitis C, comparable and considerable levels of PPARγ in the liver (FIG. 1).

The Western blotting and immunohistochemistry studies give comparable results demonstrating the expression of the PPARγ protein, in particular in the hepatocytes. The same type of study is being undertaken in order to demonstrate the expression of PPARα, -β, -δ and of LXR, TRs, BAR/FXR and SXR/PXR. The presence of large amounts of these receptors in the liver implies that modulators of PPARα, -β, -δ and -γ and of LXR, TRs, BAR/FXR and SXR/PXR will be active in the liver.

3. Modulation of the Hepatocyte Expression of CD36 by the Nuclear Receptors

By using hepatocyte lines (HepG2, HuH7) maintained in culture with agonists and/or antagonists of PPARα, -β, and -δ, and of LXR, TRs, BAR/FXR and SXR/PXR, we have demonstrated, by immunohistochemistry, respectively an increase and a decrease in the expression of CD36 by the hepatocytes. These results are in the process of being confirmed by other PCR, Western blotting and flow cytometry techniques.

In parallel, liver samples were taken from wild-type mice and from mice knocked out for the various isoforms of PPAR and for LXR, TRs, BAR/FXR and SXR/PXR. Quantification of the CD36 in these various models also demonstrates the involvement, in vivo, of these nuclear receptors in the expression of CD36 in the liver. Similarly, the use of modulators for these nuclear receptors will enable the inventors to control, in wild-type animals, their roles on the expression of CD36 in the liver.

4. Anti-Inflammatory and Anti-Fibrotic Roles of the Nuclear Receptors in the Liver

Given that no animal model other than the monkey can be infected with the hepatitis C virus, and, a fortiori, develop viral hepatitis C, forms of hepatitis induced by chemical agents (carbon tetrachloride) are used to demonstrate the anti-inflammatory and anti-fibrotic effect of modulators of the PPAR, LXR, TR, BAR/FXR and SXR/PXR nuclear receptors. The first macroscopic results reflect the anti-inflammatory effect of these various modulators.

5. Activation of PPRE by the Hepatitis C Virus or the Blood of Patients Contaminated with the Hepatitis C Virus

Hepatocytes are transfected with a reporter gene (luciferase) under the control of a responsive element (PPRE, LXRE, etc.) for the receptors described above. Visualization of the activation of the reporter gene by the virus will reveal the involvement of PPAR or of the other receptors in the course of the infection with the hepatitis C virus.

6. Role of Fenofibrates, which are an Agonist of PPARα, on the Severity of Viral Hepatitis C

The role of fenofibrates on the severity of liver damage will be analyzed in the group of patients followed by the inventors at the CHRU [University Hospital] in Lille. The role of these PPARα agonists will be evaluated with respect to biological and histological criteria.

7. Expression of the Nuclear Receptors During Infection with HCV

Using the RT-PCR technique, the inventors quantified the RXRα, VDR, LXRα, PPARα, PPARδ and PPARγ messenger RNAs in liver samples taken from patients suffering from chronic hepatitis C and from control healthy individuals.

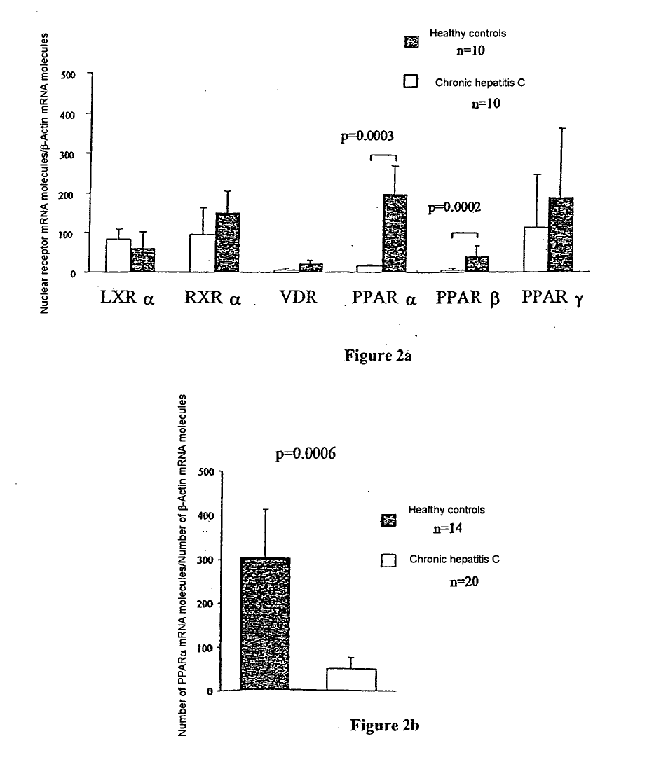

While similar concentrations of β-actin messenger RNAs are observed everywhere in the two groups tested, the level of expression of the PPARα mRNA is particularly decreased (15±1.8 versus 198±35.5, p=0.0003) and similarly for PPARβ (6±2.25 versus 40±14, p=0.0002), and a tendency for the level of expression of the PPARγ mRNA to decrease is also observed in the liver of patients infected with HCV compared to healthy controls (FIG. 2A). The PPARα concentration deficit in the liver is confirmed using the competitive RT-PCR technique (51±25.7 versus 304±110, p=0.0006) (FIG. 2B).

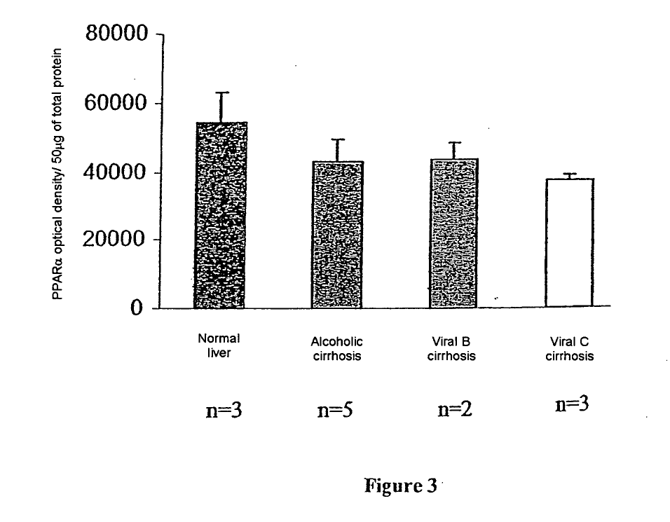

In order to analyze further along the abnormal level of expression of PPARα mRNA in the liver of patients infected with HCV, the inventors used Western blotting analysis to quantify the PPARα protein in the same samples. The inventors carried out Western blotting using an antibody directed against PPARα in the liver of control individuals and of patients infected with HCV. Given the ethical considerations, the inventors used explanted livers taken from patients who were undergoing a liver transplant because they were suffering from cirrhosis associated with an HCV infection. Using this analysis, the optical density (OD) for livers infected with HCV appears to be less intense than that for control livers (FIG. 3), suggesting that the expression of both the PPARα proteins and mRNAs is affected in the liver of patients infected with HCV.

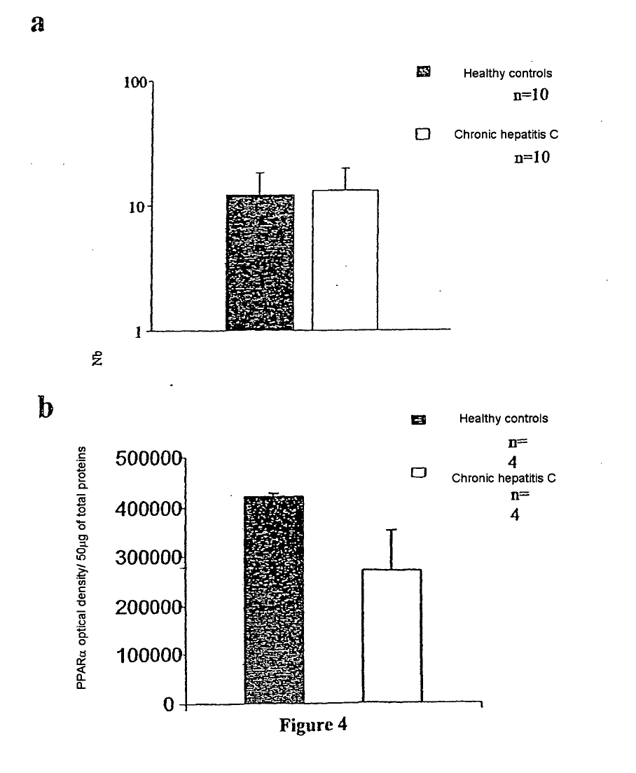

Contrary to the expression of PPARα in the PBMCs is not different in the patients infected with HCV compared to the healthy controls in terms of the mRNA levels (12.36±6.18 versus 13.15±6.57) (FIG. 4a) nor in terms of the protein (421 210±6 899 versus 270 504±82 252) (FIG. 4b). This result suggests that an abnormal expression of PPARα might be confined to the liver only and might not extend to other cells conventionally expressing PPARα.

8. Increased Susceptibility of PPARγ^{+/-} and PPARα^{-/-} Mice to CCl₄-Induced Hepatitis

As demonstrated by the previous study, the PPAR/RXR heterodimer is involved in the regulation of the inflammatory process; the inventors therefore put forward the hypothesis that the deficient expression of PPARα in the liver is liable to influence the development of inflammation after a liver injury.

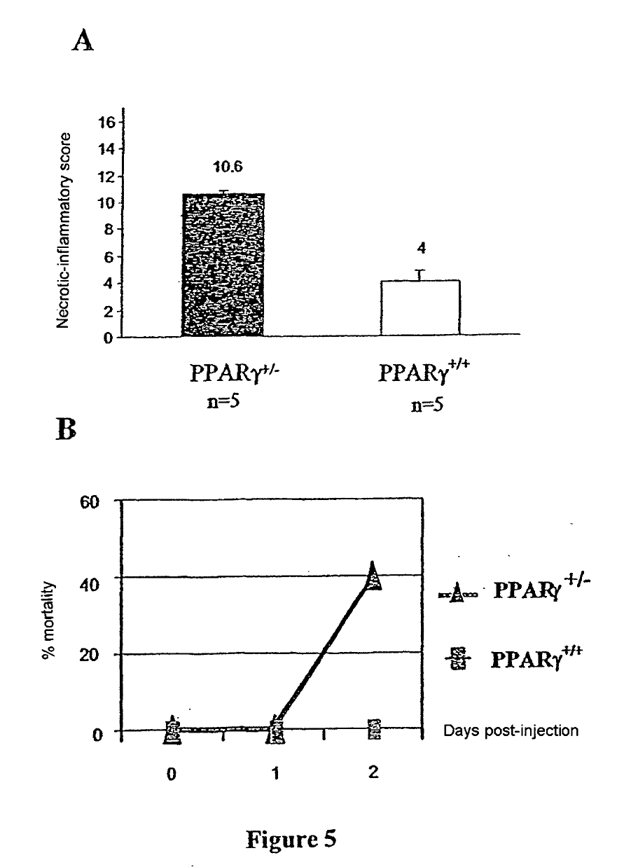

With this aim, the inventors sought to determine whether a mouse heterozygous for PPARγ deficiency (PPARγ^{+/-}) or homozygous for PPARγ deficiency (PPARγ^{-/-} was more liable to develop CCl₄-induced acute hepatitis. These mice, and also the wild-type mice from the same litter, have a 129/Sv genetic background. Compared to the PPARγ^{+/+} wild-type individuals from the same litter, the PPARγ^{+/-} mice have a greater amount of pronounced microscopic liver damage, evaluated according to Ishak (10.6±0.22 versus 4±0.8) (FIG. 5a). The liver damage is characterized by a confluent necrosis at certain sites with multiple bridges linking various vascular structures. The inflammatory infiltrate is moderate, involving a few to all of the portal areas. This susceptibility to acute hepatitis is associated with a dramatically increased mortality rate (40% compared to 0%) (FIG. 5b) only 2 days after injection of CCl₄.

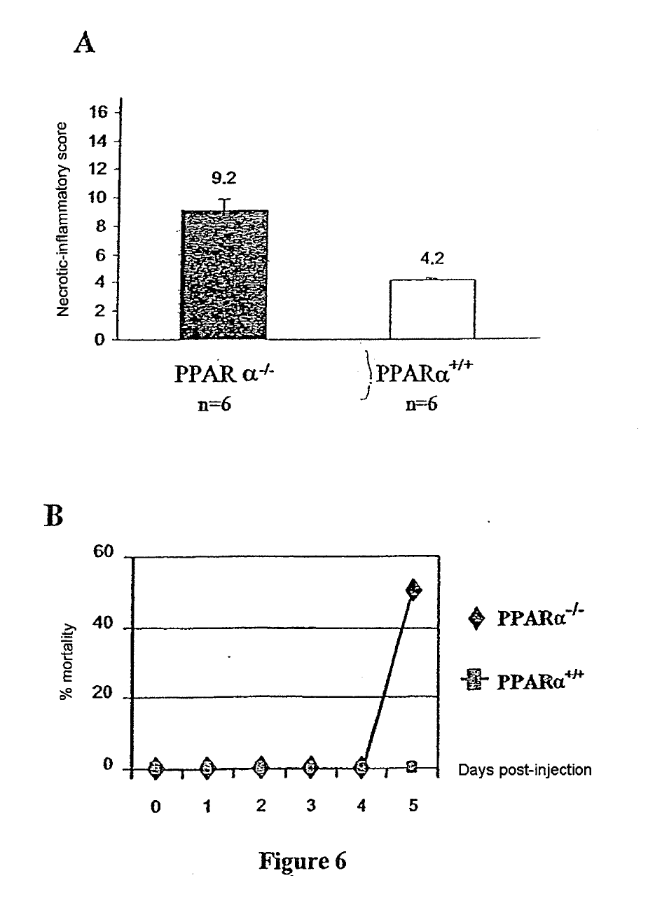

Compared to the PPARα^{+/+} wild-type individuals, the PPARα^{-/-} mice develop, 5 days after an injection with CCl₄, much more pronounced microscopic liver damage (9.2±0.7 versus 4.2±0.13) (FIG. 6a). This damage is characterized by a confluent necrosis in most of the regions and a slight inflammatory portal infiltration. However, this damage appears to be less severe than for the PPARγ^{+/-} mice. This increase in liver damage is also associated with a higher mortality rate (50% versus 0%) (FIG. 6b).

9. Disturbed PPARα mRNA Expressions in HepG2 Cells Transfected with the Core

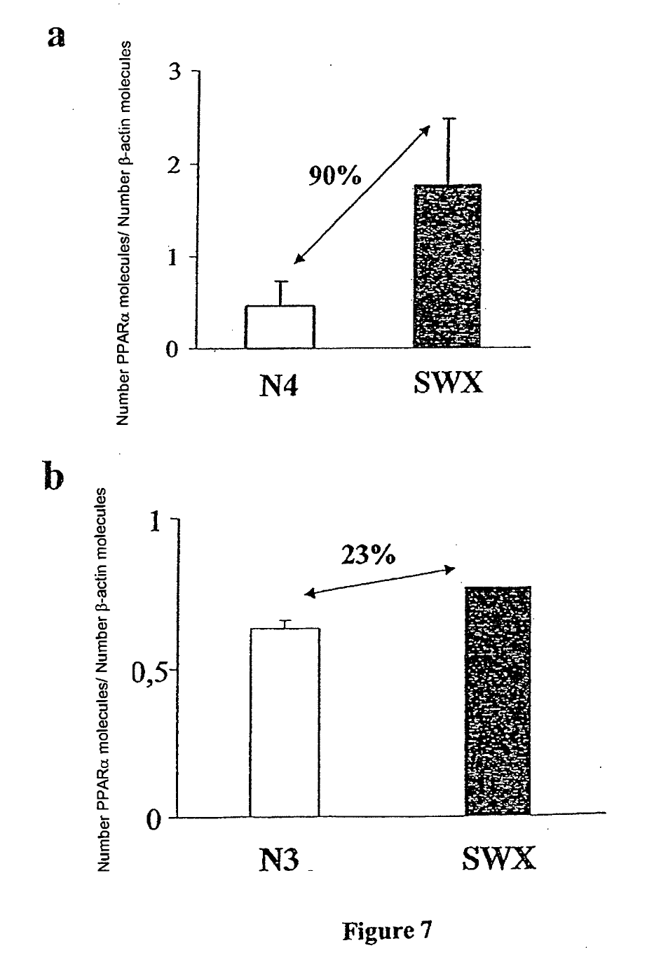

Since many studies have demonstrated the regulation of transcription of several immunoregulatory cellular genes by the HCV core, the inventors tested whether the HCV core protein could influence the expression of PPARα in transfected human hepatocyte cell lines. Using competitive RT-PCR, the inventors quantified the cellular concentration of PPARα mRNA in two independent HepG2 clones stably expressing (clone N3 and N4) or not expressing (clone SWX) the HCV core protein. Although similar concentrations of β-actin mRNA were observed in the two clones tested, a lower level of expression of PPARα messenger RNA was observed in the transfected clones compared with the SWX clones. The average rates of inhibition of the PPARα expression are 90% for the N4 clone (FIG. 7a) and 23% for the N3 clone (FIG. 7b) compared with the corresponding control SWX cells.

Thus, these results suggest that the abnormal expression of PPARα mRNA observed during chronic hepatitis C may be the result of a direct or indirect interaction between the HCV core protein and PPARα.

10. CCl₄-Induced Acute Hepatitis is Improved by PPARγ Agonists

The inventors characterized the development of acute hepatitis in Balb/c mice subjected to treatment with CCl₄, while the control mice killed three days after administration of mineral oil exhibited no microscopic damage in the liver. Three days after the induction of hepatitis, the inventors evaluated the activation of PPARγ on CCl₄-induced hepatitis using pioglitazone (50 μg/kg/day) preventively. Compared with the mice not receiving the preventive treatment, the mortality rate associated with hepatitis is decreased (40% compared to 0%) in the animals which received pioglitazone three days after treatment with CCl₄.

### References

Alter M, et al. The natural history of community-acquired hepatitis C in the United States. N Engl J Med 1992; 327: 1899-1905.

Cerny A, et al. Pathogenesis of chronic hepatitis C: immunological features of hepatic injury and viral persistence. Hepatology 1999; 30: 595-601.

Chawla A, et al. PPAR-γ dependent and independent effects on macrophage-gene expression in lipid metabolism and inflammation. Nat Med 2001; 7: 48-52.

Chinetti G, et al. PPAR-α and PPAR-γ activators induce cholesterol removal from human macrophage foam cells through stimulation of the ABCA1 pathway. Nat Med 2001; 7: 53-8.

Clouston A D, et al. Steatosis and chronic hepatitis C: analysis of fibrosis and stellate cell activation. J Hepatol 2001; 34: 314-20.

Czaja A, et al. Host- and disease-specific factors affecting steatosis in chronic hepatitis. J Hepatol 1998; 29: 198-206.

Goodman Z D, et al. Histopathology of hepatitis C virus infection. Semin Liver Dis 1995; 15: 70-81.

Knobler H, et al. Increased risk of type 2 diabetes in noncirrhotic patients with chronic hepatitis C virus infection. Mayo Clin Proc 2000; 75: 355-9.

Kuo G, et al. An assay for circulating antibodies to a major etiologic virus of human non-A, non-B hepatitis. Science 1989; 244: 362-4.

Lehmann J M, et al. An antidiabetic thiazolidinedione is a high affinity ligand for peroxysome proliferator-activated receptor gamma (PPAR gamma). J Biol Chem 1995; 270: 12953-6.

McGuinness P H, et al. Increases in hepatic CD68 positive cells, and proinflammatory cytokines (particularly interleukin 18) in chronic hepatitis C infection. Gut 2000; 46: 260-9.

McHutchison J, et al. Interferon alpha-2b alone or in combination with ribavirin for the treatment as initial treatment for chronic hepatitis C. International hepatitis interventional therapy group. N Engl J Med 1998; 339: 1493-9.

Mehta S H, et al. Prevalence of type 2 diabetes mellitus among persons with hepatitis C virus infection in the United States. Ann Intern Med 2000; 133: 592-9.

Monazahian M, et al. Low density lipoprotein receptor as a candidate receptor for hepatitis C virus. J Med Virol 1999; 57: 223-9.

Moore K J, et al. The role of PPAR-γ in macrophage differentiation and cholesterol uptake. Nat Med 2001; 7: 41-7.

Napoli J, et al. Progressive liver injury in chronic hepatitis C infection correlates with increased intrahepatic expression of Th1-associated cytokines. Hepatology 1996; 24: 759-65.

Nelson D R, et al. Activation of tumor necrosis factor-alpha system in chronic hepatitis C virus infection. Dig Dis Sci 1997; 42: 2487-94.

Poynard T, et al. Natural history of liver fibrosis progression in patients with chronic hepatitis C. Lancet 1997; 349: 825-32.

Roudot-Thoraval F, et al. Epidemiological factors affecting the severity of hepatitis C virus-related liver disease: a French survey of 6 664 patients. Hepatology 1997; 26: 485-90.

Sambrook, J., Fritsch, E. F., and Maniatis (1989), T. Sec. Ed. Cold Spring Harbor Lab., Cold Spring Harbor, N.Y.

Sangiorgio L, et al. Increased frequency of HCV and HBV infection in type 2 diabetic patients. Diabetes Res Clin Pract 2000; 48: 147-51.

Shoonjans K, et al. The peroxisome proliferator activated receptors (PPARs) and their effects on lipid metabolism and adipocyte differentiation. Biochem Biphys Acta 1996; 1302: 93-109.

Tai D I, et al. Activation of nuclear factor κB in hepatitis C virus infection: Implications for pathogenesis and hepatocarcinogenesis. Hepatology 2000; 31: 656-64.

Tsai S L, et al. Detection of Th2-like T-helper cells in hepatitis C virus infection: Implications for hepatitis C virus chronicity. Hepatology 1997; 25: 449-58.

1. The use of a compound modulating the activity of an RXR-nuclear receptor heterodimer, for preparing a medicinal product intended for the preventive and/or curative treatment of an infection with a virus which uses, in order to perform its viral cycle, at least one cellular protein encoded by a gene which has at least one RE regulatory sequence.

2. The use as claimed in claim 1, characterized in that said RXR-nuclear receptor heterodimer is selected from the group composed of RXR-PPAR, RXR-LXR, RXR-TR, RXR-BAR/FXR and RXR-SXR/PXR.

3. The use as claimed in claim 2, characterized in that said heterodimer is RXR-PPAR.

4. The use as claimed in claims 1 to 3, characterized in that said RE regulatory sequence is selected from the group composed of the PPAR-responsive regulatory sequence PPRE, the LXR-responsive regulatory sequence LX-RE, the thyroid hormone receptor (TR)-responsive regulatory sequence TRE, the BAR/FXR-responsive regulatory sequence BAR/FXR-RE, and the SXR/PXR-responsive regulatory sequence SXR/PXR-RE.

5. The use as claimed in claim 4, characterized in that said RE regulatory sequence is PPRE.

6. The use as claimed in claims 3 to 5, characterized in that said RXR-nuclear receptor heterodimer is RXR-PPAR and said RE regulatory sequence is PPRE.

7. The use as claimed in claims 1 to 6, characterized in that said cellular protein is the low density lipoprotein receptor (LDLR or CD36).

8. The use as claimed in claims 1 to 7, characterized in that said compound activates or increases the activity of the RXR-nuclear receptor heterodimer.

9. The use as claimed in claim 8, characterized in that said compound is selected from the group composed of PPARα, PPARβ, PPARγ, PPARδ and RXR, and a ligand which is a natural or synthetic agonist of PPARα, PPARβ, PPARγ, PPARδ, RXR, RXR-PPARα, RXR-PPARβ, RXR-PPARγ or RXR-PPARδ.

10. The use as claimed in claim 9, characterized in that said ligand which is an agonist of RXR-PPARα, RXR-PPARβ, RXR-PPARγ or RXR-PPARδ is chosen from the group composed of the J2 prostaglandins, polyunsaturated fatty acids, thiazolinediones, nonsteroidal anti-inflammatories, fibrates and pioglitazone.

11. The use as claimed in claims 1 to 7, characterized in that said compound abolishes or inhibits the activity of the RXR-nuclear receptor heterodimer.

12. The use as claimed in claims 1 to 11, characterized in that said virus is the hepatitis C virus.

13. The use as claimed in claim 12, for preparing a medicinal product intended for the preventive and/or curative treatment of an infection, with the hepatitis C virus, of cells selected from hepatocytes, bile cells, liver parenchymal cells and circulating blood cells.

14. The use as claimed in claims 1 to 13, for preparing a medicinal product intended for the preventive and/or curative treatment of chronic hepatitis.

15. The use as claimed in claims 1 to 13, for preparing a medicinal product intended for the preventive and/or curative treatment of fatty liver whether or not associated with a hepatocyte infection with the hepatitis C virus.

16. The use of a compound as claimed in claims 1 to 13, for preparing a medicinal product intended for the preventive and/or curative treatment of liver inflammation and of liver damage whether or not associated with a hepatocyte infection with the hepatitis C virus.

17. The use of a compound as claimed in claims 1 to 13, for preparing a medicinal product intended for the preventive and/or curative treatment of liver cirrhosis and/or of a posthepatic cancer whether or not associated with a hepatocyte infection with the hepatitis C virus.

18. The use of a compound as claimed in claims 1 to 13, for preparing a medicinal product intended for the control of carbohydrate metabolism and/or for the preventive and/or curative treatment of type II diabetes in patients carrying the hepatitis C virus.

19. A pharmaceutical composition for the preventive and/or curative treatment of a hepatitis C virus infection, characterized in that it contains a therapeutically effective amount of a compound as defined in either of claims 9 and 10.

20. The composition as claimed in claim 19, characterized in that it also contains at least one antiviral agent as a combination product for simultaneous or separate use or use spread out over time, in antiviral therapy associated with a hepatitis C virus infection.

21. The pharmaceutical composition as claimed in claim 20, characterized in that said antiviral agent is selected from the group composed of alpha-interferon (αIFN), ribavirin and delayed interferon.
